(19) United States
(12) Patent Application Publication
(10) Pub. No.: US 2022/0037006 A1
(43) Pub. Date: Feb. 3, 2022

(54) METHOD, APPARATUS AND SYSTEM FOR DIAGNOSING STATUS OF RADIOTHERAPY EQUIPMENT AND STORAGE MEDIUM

(71) Applicants: OUR UNITED CORPORATION, Xi'an City, Shaanxi (CN); SHENZHEN OUR NEW MEDICAL TECHNOLOGIES DEVELOPMENT CO., LTD., Shenzhen (CN)

(72) Inventors: Peng ZAN, Xi'an City, Shaanxi (CN); Hao YAN, Xi'an City, Shaanxi (CN)

(21) Appl. No.: 17/276,463

(22) PCT Filed: Sep. 14, 2018
(86) PCT No.: PCT/CN2018/105665

§ 371 Date: Mar. 15, 2021

(51) Int. Cl. G16H 40/40; G16H 40/67; G06Q 10/00; G16H 40/20; G16H 50/20

(52) U.S. Cl. CPC G16H 40/40; G16H 40/67; G16H 50/20; G16H 40/20; G06Q 10/20

(57) ABSTRACT

A method for diagnosing status of radiotherapy equipment includes: acquiring real-time detection data in an operating process of radiotherapy equipment; and processing the real-time detection data by using a status diagnosis model, and generating and outputting real-time status diagnosis data, wherein the real-time status diagnosis data includes at least one of real-time fault diagnosis data and real-time aging diagnosis data.

### CROSS-REFERENCE TO RELATED APPLICATIONS

This application is a US national phase application of PCT Application No. PCT/CN2018/105665, filed on Sep. 14, 2018, the entire content of which is incorporated herein by reference.

### TECHNICAL FIELD

The present disclosure relates to the field of radiation therapy technologies, and in particular to a method, apparatus and system for diagnosing status of radiotherapy equipment, and a storage medium thereof.

### BACKGROUND

Radiation therapy (radiotherapy) is a localized treatment method using radiation to treat tumors. Radiotherapy equipment may generally include a plurality of components such as a radiotherapy gantry, a radiation source, a collimator, a treatment couch, and an imaging apparatus.

### SUMMARY

The present disclosure provides a method, apparatus and system for diagnosing status of radiotherapy equipment, and a storage medium thereof. The technical solutions are as follows:

In one aspect, a method for diagnosing status of radiotherapy equipment is provided. The method includes:

acquiring real-time detection data in an operating process of the radiotherapy equipment;

generating real-time status diagnosis data by processing the real-time detection data by using a status diagnosis model, wherein the real-time status diagnosis data includes at least one of real-time fault diagnosis data and real-time aging diagnosis data; and

outputting the real-time status diagnosis data.

In some embodiments, before acquiring the real-time detection data in the operating process of the radiotherapy equipment, the method further includes:

acquiring a plurality of pieces of training sample data, wherein each of the plurality of pieces of training sample data includes a set of detection data and a corresponding set of status diagnosis data, and the status diagnosis data includes at least one of fault diagnosis data and aging diagnosis data; and

acquiring the status diagnosis model by deep learning (DL) on the acquired plurality of pieces of training sample data.

In some embodiments, the method further includes:

receiving revised training sample data, wherein the revised training sample data includes revised data acquired by revising the real-time status diagnosis data and the real-time detection data corresponding to the real-time status diagnosis data;

setting a weight value of the revised training sample data to be greater than a predetermined weight value; and

updating the status diagnosis model by deep learning on the plurality of pieces of training sample data and the revised training sample data.

In some embodiments, the radiotherapy equipment includes various types of components; and acquiring the status diagnosis model by deep learning on the acquired plurality of pieces of training sample data includes:

acquiring training sample data of each component in at least one component by classifying the plurality of pieces of training sample data based on a type of a component corresponding to detection data in each of the plurality of pieces of training sample data; and

acquiring a status diagnosis model of the each component by deep learning on the training sample data of each component; and

in response to acquiring real-time detection data of a target component, generating the real-time status diagnosis data by processing the real-time detection data by using the status diagnosis model includes:

generating the real-time status diagnosis data by processing the real-time detection data of the target component by using a status diagnosis model of the target component.

In some embodiments, acquiring the status diagnosis model by deep learning on the acquired plurality of pieces of training sample data includes:

classifying the plurality of pieces of training sample data based on a type of detection data in each of the plurality of pieces of training sample data; and

acquiring a plurality of types of status diagnosis models by deep learning on each type of training sample data; and

generating the real-time status diagnosis data by processing the real-time detection data by using the status diagnosis model includes:

determining a corresponding type of status diagnosis model based on a type of the acquired real-time detection data; and

generating the real-time status diagnosis data by processing the real-time detection data by using the corresponding type of status diagnosis model.

In another aspect, a system for diagnosing status of radiotherapy equipment is provided. The system includes:

radiotherapy equipment;

a detection apparatus, disposed in the radiotherapy equipment, and configured to acquire detection data in real time in an operating process of the radiotherapy equipment;

a status diagnosis server, connected to the detection apparatus, and configured to acquire the real-time detection data acquired by the detection apparatus, and generate and output real-time status diagnosis data by processing the real-time detection data by using a status diagnosis model, wherein the real-time status diagnosis data includes at least one of real-time fault diagnosis data and real-time aging diagnosis data; and

a remote maintenance platform, connected to the status diagnosis server, and configured to receive and display the real-time status diagnosis data, to instruct a maintainer to maintain the radiotherapy equipment based on the real-time status diagnosis data.

Optionally, the status diagnosis server is further configured to:

acquire a plurality of pieces of training sample data, wherein each of the plurality of pieces of training sample data comprises a set of detection data and a corresponding set of status diagnosis data, and the status diagnosis data includes at least one of fault diagnosis data and aging diagnosis data; and

acquire the status diagnosis model by deep learning on the acquired plurality of pieces of training sample data.

In some embodiments, the status diagnosis server is further configured to:

receive revised training sample data, wherein the revised training sample data includes revised data acquired by revising the real-time status diagnosis data and the real-time detection data corresponding to the real-time status diagnosis data;

set a weight value of the revised training sample data to be greater than a predetermined weight value; and

update the status diagnosis model by deep learning on the plurality of pieces of training sample data and the revised training sample data.

In some embodiments, the radiotherapy equipment includes various types of components; and acquiring, by the status diagnosis server, the status diagnosis model by deep learning on the acquired plurality of pieces of training sample data includes:

acquiring training sample data of each component in at least one component by classifying the plurality of pieces of training sample data based on a type of a component corresponding to detection data in each of the plurality of pieces of training sample data; and

acquiring a status diagnosis model of the each component by deep learning on the training sample data of each component; and

in response to acquiring real-time detection data of a target component, generating, by the status diagnosis server, the real-time status diagnosis data by processing the real-time detection data by using the status diagnosis model includes:

generating the real-time status diagnosis data by processing the real-time detection data of the target component by using a status diagnosis model of the target component.

In some embodiments, acquiring, by the status diagnosis server, the status diagnosis model by deep learning on the acquired plurality of pieces of training sample data includes:

classifying the plurality of pieces of training sample data based on a type of detection data in each of the plurality of pieces of training sample data; and

acquiring a plurality of types of status diagnosis models by deep learning on each type of training sample data; and

generating the real-time status diagnosis data by processing the real-time detection data by using the status diagnosis model includes:

determining a corresponding type of status diagnosis model based on a type of the acquired real-time detection data; and

generating the real-time status diagnosis data by processing the real-time detection data by using the corresponding type of status diagnosis model.

In some embodiments, the radiotherapy equipment is disposed at a radiotherapy center, and the remote maintenance platform is disposed at an equipment maintenance center; and the status diagnosis server is disposed at one of the radiotherapy center, the equipment maintenance center, and a cloud computing center.

In still another aspect, an apparatus for diagnosing status of radiotherapy equipment is provided. The apparatus includes:

an acquiring module, configured to acquire real-time detection data in an operating process of the radiotherapy equipment;

a processing module, configured to generate real-time status diagnosis data by processing the real-time detection data by using a status diagnosis model, wherein the real-time status diagnosis data includes at least one of real-time fault diagnosis data and real-time aging diagnosis data; and

an outputting module, configured to output the real-time status diagnosis data.

In yet another aspect, a non-transitory computer-readable storage medium storing at least one instruction is provided, wherein the non-transitory computer-readable storage medium, when running on a computer, causes the computer to perform the methods for diagnosing status of radiotherapy equipment in the above aspects.

It should be understood that the foregoing general description and the following detailed description are only explanatory and provide examples, and cannot limit the present disclosure.

### BRIEF DESCRIPTION OF THE DRAWINGS

For clearer descriptions of the technical solutions in the embodiments of the present disclosure, the following briefly introduces the accompanying drawings required for describing the embodiments. Apparently, the accompanying drawings in the following description show merely some embodiments of the present disclosure, and persons of ordinary skill in the art may still derive other drawings from these accompanying drawings without creative efforts.

FIG. 1 is a schematic structural diagram of a system for diagnosing status of radiotherapy equipment according to an embodiment of the present disclosure;

FIG. 2 is a flowchart of a method for diagnosing status of radiotherapy equipment according to an embodiment of the present disclosure;

FIG. 3 is a flowchart of another method for diagnosing status of radiotherapy equipment according to an embodiment of the present disclosure;

FIG. 4 is a schematic structural diagram of another system for diagnosing status of radiotherapy equipment according to an embodiment of the present disclosure;

FIG. 5 is a schematic structural diagram of still another system for diagnosing status of radiotherapy equipment according to an embodiment of the present disclosure;

FIG. 6 is a schematic structural diagram of yet another system for diagnosing status of radiotherapy equipment according to an embodiment of the present disclosure; and

FIG. 7 is a schematic structural diagram of an apparatus for diagnosing status of radiotherapy equipment according to an embodiment of the present disclosure.

Specific embodiments of the present disclosure are shown in the accompanying drawings above, and are further described in detail below. These accompanying drawings and literal description are not used to limit the scope of the conception of the present disclosure in any manner. Instead, the conception of the present disclosure is described for persons skilled in the art with reference to specific embodiments.

### DETAILED DESCRIPTION

For clearer descriptions of the objectives, technical solutions, and advantages of the present disclosure, embodiments of the present disclosure are described in detail hereinafter with reference to the accompanying drawings.

Because various components in radiotherapy equipment are mostly precision parts. When a fault occurs in the radiotherapy equipment, on-site inspection and repair by professional maintenance persons are required, and as a result the efficiency of detection and repair is relatively low.

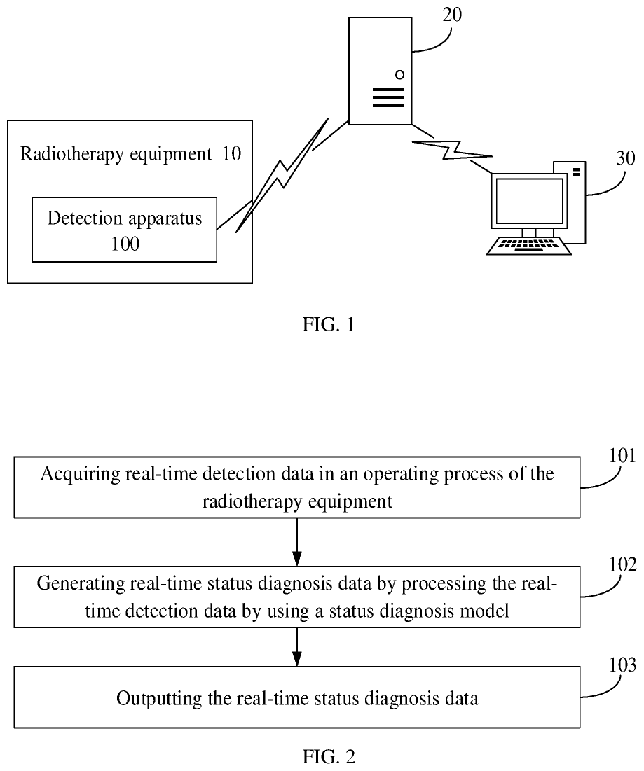

FIG. 1 is a schematic structural diagram of a system for diagnosing status of radiotherapy equipment according to an embodiment of the present disclosure. As shown in FIG. 1, the system may include radiotherapy equipment 10 and a detection apparatus 100. The detection apparatus 100 is disposed in the radiotherapy equipment 10. The system may further include a status diagnosis server 20 and a remote maintenance platform 30. The status diagnosis server 20 is connected to the detection apparatus 100, and the remote maintenance platform 30 is connected to the status diagnosis server 20.

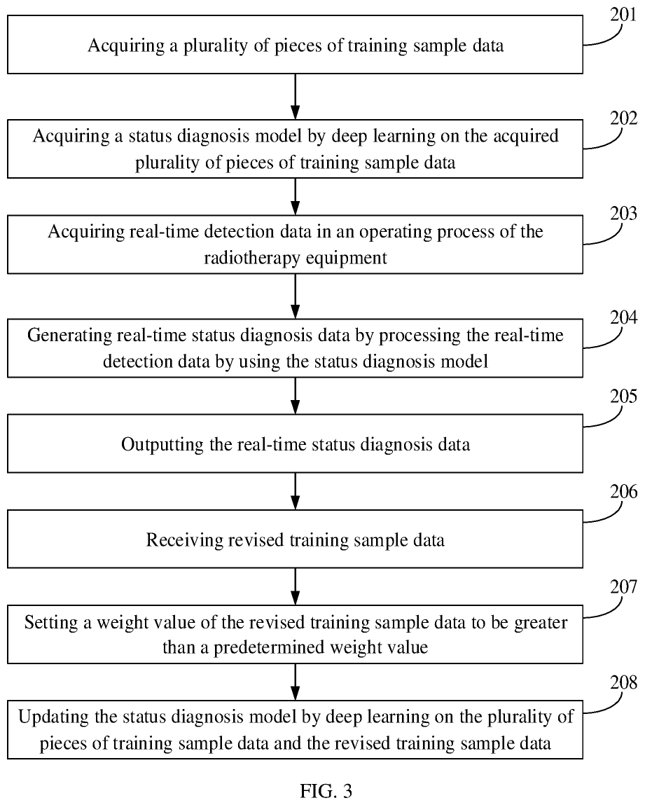

FIG. 2 is a flowchart of a method for diagnosing status of radiotherapy equipment according to an embodiment of the present disclosure. The method may be applicable to the status diagnosis server 20 in the system for diagnosing a status shown in FIG. 1. Referring to FIG. 2, the method may include the following processes.

In 101, real-time detection data in an operating process of the radiotherapy equipment is acquired.

In the operating process of the radiotherapy equipment, the detection apparatus 100 may acquire real-time detection data of various components in the radiotherapy equipment 10 in real time, and report the real-time detection data to the status diagnosis server 20 in real time. The status diagnosis server 20 acquires the real-time detection data acquired by the detection apparatus 100.

In 102, real-time status diagnosis data is generated by processing the real-time detection data by using a status diagnosis model.

The status diagnosis server 20 processes the real-time detection data by using the status diagnosis model, and generates the real-time status diagnosis data. The status diagnosis model may be acquired by training a large amount of training sample data by the status diagnosis server 20 in advance. The real-time status diagnosis data may include at least one of real-time fault diagnosis data and real-time aging diagnosis data.

In 103, the real-time status diagnosis data is outputted.

In the embodiments of the present disclosure, the status diagnosis server 20 may output the real-time status diagnosis data to the remote maintenance platform 30, for the remote maintenance platform 30 to display the real-time status diagnosis data. Alternatively, a display apparatus may also be disposed in the status diagnosis server 20, and thus the status diagnosis server 20 may output the real-time status diagnosis data to the display apparatus for display. A maintenance person may maintain the radiotherapy equipment in time based on the status diagnosis data.

In summary, the embodiments of the present disclosure provide a method for diagnosing status of radiotherapy equipment. In the method, real-time detection data in an operating process of radiotherapy equipment may be acquired, and a status diagnosis model may be used to process the real-time detection data in the operating process of radiotherapy equipment and generate real-time status diagnosis data, wherein the real-time status diagnosis data may include at least one of real-time fault diagnosis data and real-time aging diagnosis data. Therefore, when a fault occurs in the radiotherapy equipment, the status diagnosis model may be directly used to output the real-time status diagnosis data, thus the real-time status diagnosis data can be acquired without on-site inspection by a maintenance person, thereby effectively improving the efficiency of detection and repair for the radiotherapy equipment. In addition, the method for diagnosing status of radiotherapy equipment according to the embodiments of the present disclosure can implement the real-time detection of the operation status of the radiotherapy equipment, thereby effectively improving the reliability of the operation of the radiotherapy equipment.

FIG. 3 is a flowchart of another method for diagnosing status of radiotherapy equipment according to an embodiment of the present disclosure. The method may be applicable to the status diagnosis server 20 in the system for diagnosing a status shown in FIG. 1. Referring to FIG. 3, the method may include the following processes.

In 201, a plurality of pieces of training sample data are acquired.

Each of the plurality of pieces of training sample data may include a set of detection data acquired by the detection apparatus 100 and a corresponding set of status diagnosis data. The status diagnosis data may include at least one of fault diagnosis data and aging diagnosis data, and the status diagnosis data may be data verified by a maintenance person.

In some embodiments, when the status diagnosis data includes fault diagnosis data, the detection data in the plurality of pieces of training sample data may include detection data acquired by performing detection on a plurality of pieces of radiotherapy equipment when faults occur in the radiotherapy equipment. When the status diagnosis data includes aging diagnosis data, the detection data in the plurality of pieces of training sample data may include detection data acquired by performing detection on a plurality of pieces of radiotherapy equipment within the entire life cycle of the radiotherapy equipment (that is, from the moment at which the radiotherapy equipment starts to be used to the moment at which the life cycle ends). The fault diagnosis data may include data such as fault causes, actual repair solutions used for repairing the radiotherapy equipment (if the fault is caused together by a plurality of components, the repair solution involves a solution of repairing or adjusting the plurality of components), repair drawings, and maintenance opinions for maintaining the radiotherapy equipment. The aging diagnosis data may include data such as an aging degree, the remaining service times, and the remaining life cycles of the radiotherapy equipment. The fault diagnosis data and the aging diagnosis data may both be provided by a maintenance person.

In 202, a status diagnosis model is acquired by deep learning on the acquired plurality of pieces of training sample data.

The status diagnosis server 20 may acquire the status diagnosis model by training the acquired plurality of pieces of training sample data by using a deep learning method. For example, the status diagnosis model may be acquired by training the plurality of pieces of training sample data by using a convolutional neural network (CNN).

When the status diagnosis data includes the fault diagnosis data and the aging diagnosis data, the status diagnosis server 20 may acquire fault training sample data and aging training sample data by further classifying the plurality of pieces of training sample data. The detection data in the fault training sample data is detection data acquired by detection on the plurality of pieces of radiotherapy equipment when faults occur in the radiotherapy equipment. Correspondingly, the status diagnosis data is fault diagnosis data. The detection data in the aging training sample data is detection data acquired by detection on a plurality of pieces of radiotherapy equipment during an entire life cycle of the radiotherapy equipment. Correspondingly, the status diagnosis data is aging diagnosis data.

In response to deep learning on the training sample data, the status diagnosis server 20 may separately perform deep learning on the fault training sample data and the aging training sample data. Therefore, the status diagnosis model eventually acquired by the status diagnosis server 20 may include a fault diagnosis model and an aging diagnosis model. The fault diagnosis model may be configured to generate real-time fault diagnosis data in response to processing real-time detection data. The aging diagnosis model may be configured to generate real-time aging diagnosis data in response to processing the real-time detection data.

In some embodiments, in an operating process of the radiotherapy equipment, the detection data acquired by the detection apparatus 100 is multiple, and there may be some invalid data which is not closely correlated to equipment faults or equipment aging. Therefore, to improve the training efficiency of the status diagnosis model, prior to deep learning on the plurality of pieces of training sample data, operators may further label valid data in the plurality of pieces of training sample data. Correspondingly, the status diagnosis server 20 may perform deep learning only on the labeled valid data.

In some embodiments, the radiotherapy equipment may include a plurality of components, for example, may include at least one component of a radiotherapy gantry, a radiation source, a collimator, a treatment couch, a slip ring, and an imaging apparatus. The process 202 may include the following sub-processes.

In 2021a, training sample data of each component in at least one component is acquired by classifying the plurality of pieces of training sample data based on a type of a component corresponding to detection data in each of the plurality of pieces of training sample data.

In some embodiments, the status diagnosis server 20 may acquire training sample data of the radiotherapy gantry, training sample data of the radiation source, training sample data of the collimator, training sample data of the treatment couch, training sample data of the slip ring, and training sample data of the imaging apparatus by classifying the detection data acquired by the detection apparatus 100.

In 2022a, a status diagnosis model of each component is acquired by deep learning on the training sample data of each component separately.

In some embodiments, in response to deep learning on the various classes of training sample data, the status diagnosis server 20 may acquire a status diagnosis model of the radiotherapy gantry, a status diagnosis model of the radiation source, a status diagnosis model of the collimator, a status diagnosis model of the treatment couch, a status diagnosis model of the slip ring, and a status diagnosis model of the imaging apparatus.

Since different components in the radiotherapy equipment have different working principles, precision degrees, service environments, and life cycles, a status diagnosis model for each component is generated based on training sample data of each component, and real-time detection data is processed based on a corresponding status diagnosis model, such that the reliability of real-time status diagnosis data generated by the status diagnosis model is effectively improved.

In some embodiments, the detection data in the training sample data and the real-time detection data may both include a plurality of types of data, and for example, may include: at least one of current, voltage, displacement, temperature, and pressure. The process 202 may include the following sub-processes.

In 2021b, the plurality of pieces of training sample data are classified based on a type of detection data in each of the plurality of pieces of training sample data.

When classifying the plurality of pieces of training sample data, the status diagnosis server 20 may, based on the types of detection data, classify each type of detection data into one class, or classify similar types of detection data into one class.

In some embodiments, it is assumed that the detection data in the plurality of pieces of training sample data acquired by the status diagnosis server 20 includes current, voltage, displacement, and temperature. The status diagnosis server 20 may classify the plurality of pieces of training sample data into four classes. The four classes of training sample data include: training sample data of a current class, training sample data of a voltage class, training sample data of a displacement class, and training sample data of a temperature class. Alternatively, because the current and voltage have similar types, the status diagnosis server 20 may classify the training sample data of the current class and the training sample data of the voltage class into one same class.

In 2022b, a plurality of types of status diagnosis models are acquired by deep learning on each type of training sample data.

In some embodiments, the status diagnosis server 20 may acquire a status diagnosis model of the current class, a status diagnosis model of the voltage class, a status diagnosis model of the displacement class, and a status diagnosis model of the temperature class by deep learning on the four classes of training sample data.

In 203, real-time detection data in an operating process of radiotherapy equipment is acquired.

In the operating process of the radiotherapy equipment, the detection apparatus 100 may acquire real-time detection data of various components in real time, and may report the real-time detection data to the status diagnosis server 20. The types of real-time detection data may be different depending on the type of the detection apparatus 100.

In some embodiments, the real-time detection data may include at least one of a current acquired by an ammeter, a voltage acquired by a voltmeter, a displacement detected by a displacement sensor, a temperature acquired by a temperature sensor, and a pressure acquired by a pressure sensor.

In 204, the real-time detection data is processed by using the status diagnosis model, and real-time status diagnosis data is generated.

Further, the status diagnosis server 20 may process, by using the status diagnosis models acquired by training in advance, the real-time detection data acquired from the detection apparatus 100, and generate the real-time status diagnosis data. The type of data included in the real-time status diagnosis data may be consistent with the type of data included in the status diagnosis data in the training sample data used during deep learning.

In some embodiments, the imaging apparatus is used as an example. The imaging apparatus is a part that is located in the radiotherapy equipment and is configured to perform image scanning on the lesion of patients. The core element of the imaging apparatus is a tube (for example, an X-ray tube). After the tube has been used for a long time, the aging of the sealing material of the tube would cause a loss in the vacuum of the tube, and an arcing phenomenon would occur in the tube. If the arcing phenomenon occurs frequently, the radiotherapy equipment may fail to work normally. The arcing phenomenon is further accompanied with a sudden rise in the voltage in the tube. In severe cases, the voltage in the tube exceeds a predetermined voltage value to cause overload, and as a result the filament in the tube breaks or the high-voltage power-supply circuit trips. When the arcing phenomenon occurs frequently in the tube, the tube fails to achieve exposure, and as a result the radiotherapy equipment fails to operate normally. In this case, a professional maintenance person needs to perform equipment detection on the radiotherapy equipment on site, to detect whether the tube fails to cause the radiotherapy equipment to fail to operate normally. In addition, the maintenance person further needs to repair the tube or discard and replace an unrepairable tube.

In the embodiments of the present disclosure, a detection module may acquire the voltage of a plurality of tubes within their entire life cycles. The maintenance person may label abnormal voltage when a fault occurs in a tube and corresponding fault diagnosis data, and may label voltages in different periods within the entire life cycle of the tube and aging degrees of the corresponding periods. Subsequently, the maintenance person may input the labeled data as training sample data into the status diagnosis server 20. The status diagnosis server 20 may acquire a status diagnosis model of the tube by training the acquired training sample data by using a deep learning algorithm. Further, in an actual operating process of the radiotherapy equipment, the detection apparatus 100 may acquire real-time voltage of the tube in real time, and send the real-time voltage to the status diagnosis server 20. The status diagnosis server 20 may acquire the fault diagnosis data and the aging diagnosis data of the tube by processing the received real-time voltage based on the status diagnosis model.

In 205, the real-time status diagnosis data is outputted.

In the embodiments of the present disclosure, the status diagnosis server 20 may output the real-time status diagnosis data, to instruct a maintenance person to maintain the radiotherapy equipment based on the real-time status diagnosis data. For example, the status diagnosis server 20 may output the real-time status diagnosis data to the remote maintenance platform 30, for the remote maintenance platform 30 to display the real-time status diagnosis data. Alternatively, the system for diagnosing a status may further include a display apparatus. The status diagnosis server 20 may output the real-time status diagnosis data to the display apparatus for display. The display apparatus may be disposed in the status diagnosis server 20 or may be disposed in the remote maintenance platform 30.

For example, the imaging apparatus is used as an example. After the status diagnosis server 20 outputs the fault diagnosis data and the aging diagnosis data of the tube, the maintenance person may rapidly determine a repair solution based on the fault diagnosis data, and repair the tube in time. In addition, the maintenance person may further determine information such as the aging degrees, the remaining life cycle, and the remaining service times of the tube based on the aging diagnosis data, to provide pre-warnings for operators of the radiotherapy equipment, making it convenient for the operators to acknowledge the remaining life cycle of the tube in time and replace the tube in time. Further, when determining, based on the fault diagnosis data, that the tube is no longer serviceable, or determining, based on the aging diagnosis data, that the remaining life cycle of the tube is relatively short, the maintenance person may further customize and order a tube in advance, thereby effectively shortening the repair period of the radiotherapy equipment and reducing the impact on the normal operation of the radiotherapy equipment.

In some embodiments, as shown in FIG. 3, the method may further include the following processes.

In 206, revised training sample data is received.

After the status diagnosis server 20 outputs the real-time status diagnosis data, if the maintenance person determines that the accuracy of the real-time status diagnosis data is relatively low and the real-time status diagnosis data need to be revised before actually being used for equipment repair or equipment maintenance, the maintenance person may further input the revised sample data upon revision into the status diagnosis server 20. The revised training sample data may include revised data acquired by revising the real-time status diagnosis data and real-time detection data corresponding to the real-time status diagnosis data.

In some embodiments, when the maintenance person cannot repair a fault based on the fault diagnosis data outputted by the status diagnosis server 20, the maintenance person may further acquire the detection data acquired by the detection apparatus 100, and determine a fault cause and a repair solution based on the detection data and perform repair. Correspondingly, the maintenance person may input the re-determined repair solution and the detection data as the revised training sample data into the status diagnosis server 20.

For example, the tube is used as an example. After acquiring the fault diagnosis data generated by the status diagnosis server 20, if the repair solution in the fault diagnosis data need to be revised before actually being used to repair the tube, the maintenance person may further re-input the revised fault diagnosis data and the corresponding abnormal voltage as the revised training sample data into the status diagnosis server 20.

In 207, a weight value of the revised training sample data is set to be greater than a predetermined weight value.

Because the revised training sample data is the training sample data revised by the maintenance person and has relatively high reliability, the weight value of the revised training sample data may be set relatively high. The predetermined weight value may be an initial weight value allocated to each of the plurality of pieces of training sample data when the status diagnosis server 20 performs deep learning on the plurality of pieces of training sample data for the first time.

In the embodiments of the present disclosure, to make it convenient for the status diagnosis server 20 to recognize the revised training sample data, when inputting the revised training sample data, the maintenance person may add a revision identifier to the revised training sample data. The revision identifier indicates that the training sample data is training sample data that is revised by the maintenance person. In response to receiving newly added training sample data, the status diagnosis server 20 detects that the newly added training sample data carries the revision identifier, and hence determines that the newly added training sample data is revised training sample data, and sets a weight value of the newly added training sample data to be greater than the predetermined weight value.

In 208, the status diagnosis model is updated by deep learning the plurality of pieces of training sample data and the revised training sample data.

In response to receiving the revised training sample data, the status diagnosis server 20 may perform deep learning on the training sample data and the revised training sample data again based on the weight value of the revised training sample data, such that the status diagnosis model can be updated and completed, and the reliability of the status diagnosis model can be further improved.

In addition, if the real-time status diagnosis data generated by the status diagnosis server 20 is verified by the maintenance person and is directly used for equipment repair or equipment maintenance without revision, it indicates that the status diagnosis model is already relatively complete. Therefore, the maintenance person no longer needs to input the real-time status diagnosis data and the corresponding real-time detection data into the status diagnosis server 20. The maintenance person may input the real-time status diagnosis data and the corresponding real-time detection data into the status diagnosis server 20, to further increase a sample amount in the status diagnosis server 20.

Correspondingly, the status diagnosis server 20 may perform deep learning again on the training sample data stored in the status diagnosis server 20 in response to detecting the newly added training sample data or once every particular period, such that the status diagnosis model is being constantly optimized and perfected.

It needs to be noted that the sequence of the processes in the method for diagnosing status of radiotherapy equipment according to the embodiments of the present disclosure may be appropriately adjusted, and the processes may be added or reduced as required. Any variant method that may be readily figured out by a person skilled in the art within the technical scope disclosed in the present disclosure shall fall within the protection scope of the present disclosure. Therefore, details are not described again.

In summary, the embodiments of the present disclosure provide a method for diagnosing status of radiotherapy equipment. In the method, a status diagnosis model may be acquired by deep learning on a plurality of pieces of training sample data, and by the status diagnosis model, real-time status diagnosis data may be generated by processing real-time detection data in an operating process of the radiotherapy equipment. Therefore, when a fault occurs in the radiotherapy equipment, real-time status diagnosis data can be acquired without on-site inspection by a maintenance person, thereby effectively improving the efficiency of detection and repair of the radiotherapy equipment. In addition, the method for diagnosing status of radiotherapy equipment according to the embodiments of the present disclosure can implement the real-time detection of the operation status of the radiotherapy equipment, thereby effectively improving the reliability of the operation of the radiotherapy equipment.

An embodiment of the present disclosure provides a system for diagnosing status of radiotherapy equipment. As shown in FIG. 1, the system may include radiotherapy equipment 10 and a detection apparatus 100. The detection apparatus 100 is disposed in the radiotherapy equipment 10 and is configured to acquire detection data in real time in an operating process of the radiotherapy equipment 10.

In some embodiments, the detection apparatus 100 may include at least one of an ammeter, a voltmeter, a displacement sensor, a temperature sensor, and a pressure sensor. Correspondingly, the detection data may include at least one of a current acquired by the ammeter, a voltage acquired by the voltmeter, a displacement acquired by the displacement sensor, a temperature acquired by the temperature sensor, and a pressure acquired by the pressure sensor.

Referring to FIG. 1, the system may further include a status diagnosis server 20 and a remote maintenance platform 30. The status diagnosis server 20 is connected to the detection apparatus 100, and is configured to acquire real-time detection data acquired by the detection apparatus 100, process the real-time detection data by using a status diagnosis model, and generate and output real-time status diagnosis data to the remote maintenance platform 30. Herein, the real-time status diagnosis data includes at least one of real-time fault diagnosis data and real-time aging diagnosis data.

The remote maintenance platform 30 may be deployed in a remote maintenance server by after-sales and repair divisions of the radiotherapy equipment, and be configured to receive and display the real-time status diagnosis data, to instruct a maintenance person to maintain the radiotherapy equipment based on the real-time status diagnosis data.

In summary, the embodiments of the present disclosure provide a system for diagnosing status of radiotherapy equipment. In the system, a status diagnosis model may be used to process real-time detection data in an operating process of radiotherapy equipment and generate real-time status diagnosis data. Therefore, when a fault occurs in the radiotherapy equipment, real-time status diagnosis data can be acquired without on-site inspection by a maintenance person, thereby effectively improving the efficiency of detection and repair of the radiotherapy equipment. In addition, the system for diagnosing status of radiotherapy equipment according to the embodiments of the present disclosure can implement the real-time detection of the operation status of the radiotherapy equipment, thereby effectively improving the operation reliability of the radiotherapy equipment. Optionally, the status diagnosis server 20 may be further configured to:

receive revised training sample data, wherein the revised training sample data includes revised data acquired by revising the real-time status diagnosis data and the real-time detection data corresponding to the real-time status diagnosis data; and

set a weight value of the revised training sample data to be greater than a predetermined weight value, and update the status diagnosis model by deep learning on the plurality of pieces of training sample data and the revised training sample data.

In some embodiments, the radiotherapy equipment may include a plurality of components, for example, at least one component of a radiotherapy gantry, a radiation source, a collimator, a treatment couch, a slip ring, and an imaging apparatus. The status diagnosis server 20 may be configured to:

acquire training sample data of each component in at least one component by classifying the plurality of pieces of training sample data based on a type of a component corresponding to detection data in each of the plurality of pieces of training sample data; acquire a status diagnosis model of each component by deep learning on the training sample data of each component; and in response to acquiring real-time detection data of a target component, generate the real-time status diagnosis data by processing the real-time detection data of the target component based on a status diagnosis model of the target component.

For example, it is assumed that the detection data in the plurality of pieces of training sample data acquired by the detection apparatus 100 includes detection data acquired by performing detection on the collimator, the treatment couch, and the imaging apparatus. After the status diagnosis server 20 acquires a plurality of pieces of training sample data including the foregoing detection data, the plurality of pieces of training sample data may be classified into three classes. The three classes of training sample data include: training sample data of the collimator, training sample data of the treatment couch, and training sample data of the imaging apparatus. Further, the status diagnosis server 20 may acquire a status diagnosis model of the collimator, a status diagnosis model of the treatment couch, and a status diagnosis model of the imaging apparatus by deep learning on each class of training sample data. During actual status diagnosis, if the status diagnosis server 20 acquires the real-time detection data of the collimator acquired by the detection apparatus, the real-time status diagnosis data for the collimator may be acquired by processing the real-time detection data based on the status diagnosis model of the collimator.

Since different components in the radiotherapy equipment have different working principles, precision degrees, service environments, and life cycles, a status diagnosis model for each component is generated based on training sample data of each component, and real-time detection data is processed based on a corresponding status diagnosis model, such that the reliability of real-time status diagnosis data generated by the status diagnosis model is effectively improved.

In some embodiments, the detection data in the training sample data and the real-time detection data may both be a plurality of types of data, for example, may include: at least one of current, voltage, displacement, temperature, and pressure. The status diagnosis server 20 may be configured to:

classify the plurality of pieces of training sample data based on the type of detection data in each of the plurality of pieces of training sample data; acquire a plurality of types of status diagnosis models by deep learning on each type of training sample data; and determine a corresponding type of status diagnosis model based on the type of the acquired real-time detection data, and generate the real-time status diagnosis data by processing the detection data based on the corresponding type of status diagnosis model.

When a status diagnosis module classifies the plurality of pieces of training sample data, the status diagnosis module may, based on the types of detection data, classify each type of detection data into one class, or classify similar types of detection data into one class.

For example, it is assumed that the detection data in the plurality of pieces of training sample data acquired by the status diagnosis server 20 includes current, voltage, displacement, and temperature. The status diagnosis server 20 may classify the plurality of pieces of training sample data into four classes. The four classes of training sample data include: training sample data of a current class, training sample data of a voltage class, training sample data of a displacement class, and training sample data of a temperature class.

Alternatively, because the current and voltage have similar types, the status diagnosis server 20 may classify the training sample data of the current class and the training sample data of the voltage class into one same class.

Further, the status diagnosis server 20 may acquire a plurality of types of status diagnosis models by deep learning on each class of training sample data. During actual status diagnosis, if the real-time detection data acquired by the detection apparatus acquired by the status diagnosis server 20 is a displacement, the status diagnosis server 20 may generate the real-time status diagnosis data by processing the real-time detection data based on the status diagnosis model acquired by training based on the displacement class of training sample data.

In some embodiments, the radiotherapy equipment 10 may be disposed at a radiotherapy center. The remote maintenance platform 30 may be disposed at an equipment maintenance center. The status diagnosis server 20 may be disposed at one of the radiotherapy center, the equipment maintenance center, and a cloud computing center.

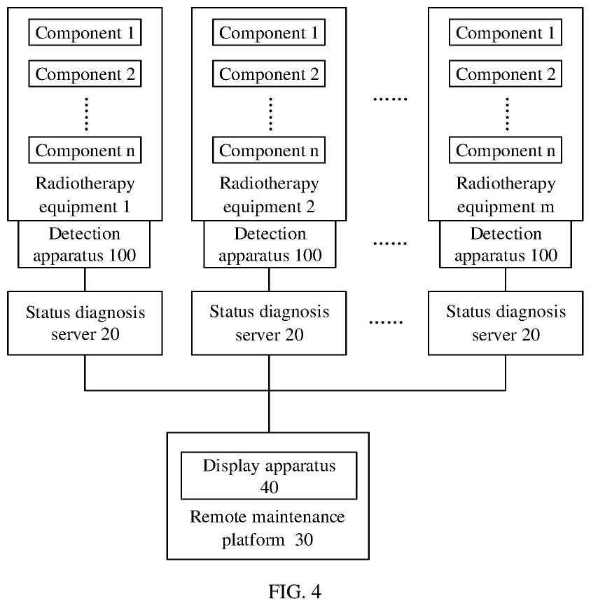

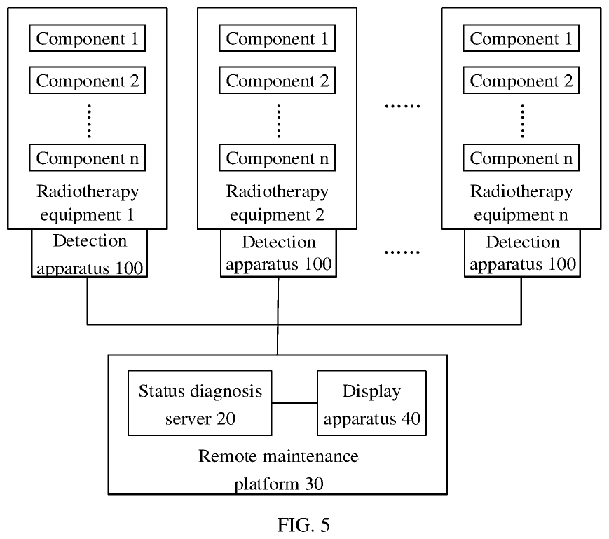

FIG. 4 is a schematic structural diagram of another system for diagnosing status of radiotherapy equipment according to an embodiment of the present disclosure. As shown in FIG. 4, the status diagnosis server 20 may be disposed on the same side as the radiotherapy equipment 10. That is, the status diagnosis server 20 and the radiotherapy equipment 10 may both be disposed at the radiotherapy center.

As can be seen from FIG. 4, the remote maintenance platform 30 may establish a communication connection with status diagnosis servers 20 disposed in a plurality of radiotherapy centers. Each status diagnosis server 20 may send real-time status diagnosis data generated by the status diagnosis server 20 to the remote maintenance platform 30.

For example, as shown in FIG. 4, the system for diagnosing status of radiotherapy equipment may perform status monitoring and diagnosis on radiotherapy equipment 1 to radiotherapy equipment m (m is an integer greater than 0) disposed at different radiotherapy centers. Each piece of radiotherapy equipment may both include a total of n components from a component 1 to a component n (n is an integer greater than 0). A detection apparatus 100 is further disposed in each piece of radiotherapy equipment, and a status diagnosis server 20 is disposed at each radiotherapy center. The status diagnosis server 20 of each radiotherapy center may be connected to the detection apparatus 100 in the radiotherapy equipment at the radiotherapy center, and may acquire the real-time detection data acquired by the detection apparatus 100, process the real-time detection data by using a status diagnosis model, and generate and output the real-time status diagnosis data.

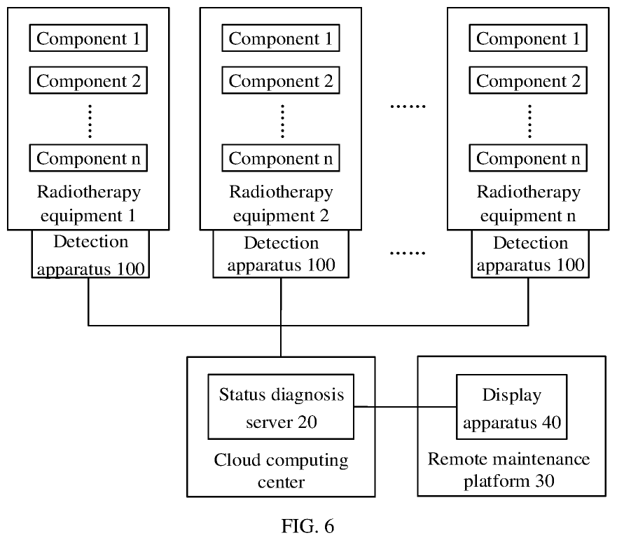

FIG. 5 is a schematic structural diagram of still another system for diagnosing status of radiotherapy equipment according to an embodiment of the present disclosure. As shown in FIG. 5, the status diagnosis server 20 may also be disposed in an equipment maintenance center in which the remote maintenance platform 30 is disposed. The status diagnosis server 20 may establish a communication connection with the detection apparatus 100 in the radiotherapy equipment disposed at each radiotherapy center, and may acquire real-time detection data acquired by the detection apparatuses 100, and generate real-time status diagnosis data of different pieces of radiotherapy equipment based on the acquired real-time detection data. The status diagnosis server 20 is disposed in the equipment maintenance center, and may acquire sufficient training sample data from the different pieces of radiotherapy equipment within a short time, thereby shortening the time of acquiring training sample data. Compared with the system shown in FIG. 4, the status diagnosis model can be generated more rapidly.

FIG. 6 is a schematic structural diagram of yet another system for diagnosing status of radiotherapy equipment according to an embodiment of the present disclosure. As shown in FIG. 6, the status diagnosis server 20 may also be disposed in the cloud computing center. The cloud computing center may be a cloud server. The status diagnosis server 20 may establish a communication connection with the detection apparatuses 100 in the radiotherapy equipment disposed at different radiotherapy centers and the remote maintenance platform 30. The status diagnosis server 20 may acquire real-time detection data acquired by the detection apparatuses 100, generate real-time status diagnosis data of different pieces of radiotherapy equipment based on the acquired real-time detection data, and send the real-time status diagnosis data to the remote maintenance platform 30. By disposing the status diagnosis server 20 in the cloud computing center, the status diagnosis server 20 is prevented from occupying computing resources at the radiotherapy center or the equipment maintenance center, effectively improving the efficiency of data processing.

Regardless of the arrangement manner of the status diagnosis module in the system for diagnosing status of radiotherapy equipment, a maintenance person from after-sales and repair divisions of the radiotherapy equipment can use the remote maintenance platform 30 to know in time real-time status diagnosis data of the radiotherapy equipment disposed in different regions, and may repair or maintain the radiotherapy equipment or order or customize in advance a replacement part based on the real-time status diagnosis data. The process does not require on-site inspection of a maintenance person, thereby greatly shortening the periods of detection and repair, and improving the efficiency of detection and repair.

In some embodiments, as seen from FIG. 4 to FIG. 6, the system for diagnosing status of radiotherapy equipment according to the embodiments of the present disclosure may further include a display apparatus 40. The status diagnosis server 20 may output the real-time status diagnosis data to the display apparatus 40. The display apparatus 40 may be configured to display the status diagnosis data.

As shown in FIG. 4 to FIG. 6, the display apparatus 40 may be disposed in the remote maintenance platform 30, such that it is convenient for a maintenance person to view the real-time status diagnosis data. The display apparatus 40 may be disposed at a radiotherapy center, such that operators at the radiotherapy center can directly check the real-time status diagnosis data.

In summary, the embodiments of the present disclosure provide a system for diagnosing status of radiotherapy equipment. A status diagnosis module in the system may acquire a status diagnosis model by deep learning on a plurality of pieces of training sample data, and the status diagnosis model may be used to process real-time detection data acquired in an operating process of radiotherapy equipment and generate real-time status diagnosis data. Therefore, when a fault occurs in the radiotherapy equipment, the status diagnosis module may directly provide real-time status diagnosis data, and on-site inspection of a maintenance person is not required, thereby effectively improving the efficiency of detection and repair. In addition, the system for diagnosing status of radiotherapy equipment according to the embodiments of the present disclosure can implement the real-time detection of the operation status of the radiotherapy equipment, thereby effectively improving the reliability of the operation of the radiotherapy equipment.

In addition, in the embodiments of the present disclosure, because the status diagnosis model used in the status diagnosis module is acquired by training based on a large amount of training sample data verified by the maintenance person, the status diagnosis model has relatively high reliability. The status diagnosis data generated by the status diagnosis model may be directly used for diagnosing status of radiotherapy equipment, or the status diagnosis data may be used for diagnosis status of radiotherapy equipment upon revision. In this way, intelligent status diagnosis is implemented, and an assistor of after-sales and repair services is caused to change from a conventional assistor (after-sales and maintenance persons of an equipment manufacturer) to a status diagnosis model. The system for diagnosing status of radiotherapy equipment, in combination with a cloud network, constitutes an artificial intelligence-based status diagnosis cloud service system.

The system for diagnosing status of radiotherapy equipment according to the embodiments of the present disclosure can monitor the entire life cycles of various parts in the radiotherapy equipment, acquire detection data of the various parts, and generate status diagnosis data of the various parts. A maintenance person may further formulate an appropriate repair plan based on aging diagnosis data in the status diagnosis data, to periodically check and maintain the various parts of the radiotherapy equipment. For example, lubrication periods of mechanical parts may be determined based on aging diagnosis data of the mechanical parts, and periodical lubrication is performed on the mechanical parts in the radiotherapy equipment based on the lubrication periods.

Alternatively, a monitoring period of a scanning apparatus may be determined based on aging diagnosis data of the scanning apparatus, and the scanning apparatus is periodically detected and maintained based on the monitoring period.

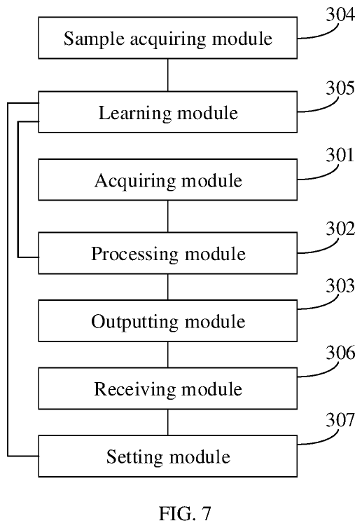

An embodiment of the present disclosure provides an apparatus for diagnosing status of radiotherapy equipment. The apparatus may be applicable to the status diagnosis server 20 in any of FIG. 1, and FIG. 4 to FIG. 6. As shown in FIG. 7, the apparatus may include:

an acquiring module 301, configured to acquire real-time detection data in an operating process of the radiotherapy equipment;

a processing module 302, configured to generate real-time status diagnosis data by processing the real-time detection data by using a status diagnosis model, wherein the real-time status diagnosis data includes at least one of real-time fault diagnosis data and real-time aging diagnosis data; and

an outputting module 303, configured to output the real-time status diagnosis data.

Optionally, as shown in FIG. 7, the apparatus may further include:

a sample acquiring module 304, configured to acquire a plurality of pieces of training sample data, wherein each of the plurality of pieces of training sample data includes a set of detection data and a corresponding set of status diagnosis data, and the status diagnosis data includes at least one of fault diagnosis data and aging diagnosis data; and

a learning module 305, configured to acquire the status diagnosis model by deep learning on the acquired plurality of pieces of training sample data.

In some embodiments, referring to FIG. 7, the apparatus may further include:

a receiving module 306, configured to receive revised training sample data, wherein the revised training sample data includes revised data acquired by revising the real-time status diagnosis data and the real-time detection data corresponding to the real-time status diagnosis data; and

a setting module 307, configured to set a weight value of the revised training sample data to be greater than a predetermined weight value.

The learning module 305 may be further configured to update the status diagnosis model by deep learning on the plurality of pieces of training sample data and the revised training sample data.

In some embodiments, the radiotherapy equipment includes various types of components; and the learning module 305 may be configured to:

acquire training sample data of each component in at least one component by classifying the plurality of pieces of training sample data based on a type of a component corresponding to detection data in each of the plurality of pieces of training sample data; and

acquire a status diagnosis model of each component by deep learning on the training sample data of each component; and

correspondingly, after the acquiring module 301 acquires the real-time detection data of a target component, the processing module 302 may be configured to:

generate the real-time status diagnosis data by processing the real-time detection data of the target component by using a status diagnosis model of the target component.

In some embodiments, the learning module 305 may be configured to:

classify the plurality of pieces of training sample data based on the type of detection data in each of the plurality of pieces of training sample data; and

acquire a plurality of types of status diagnosis models by deep learning on each type of training sample data; and

correspondingly, the processing module 302 may be configured to:

determine a corresponding type of status diagnosis model based on the type of the acquired real-time detection data; and

generate the real-time status diagnosis data by processing the real-time detection data by using the corresponding type of status diagnosis model.

In summary, the embodiments of the present disclosure provide an apparatus for diagnosing status of radiotherapy equipment. In the apparatus, a status diagnosis model may be used to process real-time detection data in an operating process of radiotherapy equipment and generate real-time status diagnosis data. Therefore, when a fault occurs in the radiotherapy equipment, real-time status diagnosis data can be acquired without on-site inspection by a maintenance person, thereby effectively improving the efficiency of detection and repair of the radiotherapy equipment. In addition, the apparatus for diagnosing status of radiotherapy equipment according to the embodiments of the present disclosure can implement the real-time detection of the operation status of the radiotherapy equipment, thereby effectively improving the operation reliability of the radiotherapy equipment.

Embodiments of the present disclosure further provide a device for diagnosing status of radiotherapy equipment. The device includes: a processor, and a memory storing a computer program runnable on the processor, wherein the processor, when running the computer program, is caused to perform the methods for diagnosing status of radiotherapy equipment according to the above method embodiments.

Embodiments of the present disclosure provide a non-transitory computer-readable storage medium storing at least one instruction, wherein the non-transitory computer-readable storage medium, when running on a computer, causes the computer to perform the methods for diagnosing status of radiotherapy equipment according to the above method embodiments.

A person of ordinary skill in the art may understand that all or a part of the processes of the embodiments may be implemented by hardware or a program instructing relevant hardware. The program may be stored in a computer-readable storage medium. The storage medium may be a read-only memory, a magnetic disk, an optical disc, or the like.

Described above are merely example embodiments of the present disclosure, and are not intended to limit the present disclosure. Within the spirit and principles of the disclosure, any modifications, equivalent substitutions, improvements, and the like are within the protection scope of the present disclosure.

1. A method for diagnosing status of radiotherapy equipment, comprising:
acquiring real-time detection data in an operating process of the radiotherapy equipment;
generating real-time status diagnosis data by processing the real-time detection data by using a status diagnosis model, wherein the real-time status diagnosis data comprises at least one of real-time fault diagnosis data and real-time aging diagnosis data; and
outputting the real-time status diagnosis data.

2. The method according to claim 1, wherein before acquiring the real-time detection data in the operating process of the radiotherapy equipment, the method further comprises:
acquiring a plurality of pieces of training sample data, wherein each of the plurality of pieces of training sample data comprises a set of detection data and a corresponding set of status diagnosis data, and the status diagnosis data comprises at least one of fault diagnosis data and aging diagnosis data; and
acquiring the status diagnosis model by deep learning on the acquired plurality of pieces of training sample data.

3. The method according to claim 2, further comprising:
receiving revised training sample data, wherein the revised training sample data comprises revised data acquired by revising the real-time status diagnosis data and the real-time detection data corresponding to the real-time status diagnosis data;
setting a weight value of the revised training sample data to be greater than a predetermined weight value; and
updating the status diagnosis model by deep learning on the plurality of pieces of training sample data and the revised training sample data.

4. The method according to claim 2, wherein
the radiotherapy equipment comprises various types of components;
acquiring the status diagnosis model by deep learning on the acquired plurality of pieces of training sample data comprises:
acquiring training sample data of each component in at least one component by classifying the plurality of pieces of training sample data based on a type of a component corresponding to detection data in each of the plurality of pieces of training sample data; and
acquiring a status diagnosis model of the each component by deep learning on the training sample data of each component; and
in response to acquiring real-time detection data of a target component, generating the real-time status diagnosis data by processing the real-time detection data by using the status diagnosis model comprises:
generating the real-time status diagnosis data by processing the real-time detection data of the target component by using a status diagnosis model of the target component.

5. The method according to claim 2, wherein
acquiring the status diagnosis model by deep learning on the acquired plurality of pieces of training sample data comprises:
classifying the plurality of pieces of training sample data based on a type of detection data in each of the plurality of pieces of training sample data; and
acquiring a plurality of types of status diagnosis models by deep learning on each type of training sample data; and
generating the real-time status diagnosis data by processing the real-time detection data by using the status diagnosis model comprises:
determining a corresponding type of status diagnosis model based on a type of the acquired real-time detection data; and
generating the real-time status diagnosis data by processing the real-time detection data by using the corresponding type of status diagnosis model.

6. A system for diagnosing status of radiotherapy equipment, comprising:
the radiotherapy equipment;
a detection apparatus, disposed in the radiotherapy equipment, and configured to acquire detection data in real time in an operating process of the radiotherapy equipment;
a status diagnosis server, connected to the detection apparatus, and configured to: acquire the real-time detection data acquired by the detection apparatus, and generate and output real-time status diagnosis data by processing the real-time detection data by using a status diagnosis model, wherein the real-time status diagnosis data comprises at least one of real-time fault diagnosis data and real-time aging diagnosis data; and
a remote maintenance platform, connected to the status diagnosis server, and configured to receive and display the real-time status diagnosis data, to instruct a maintainer to maintain the radiotherapy equipment based on the real-time status diagnosis data.

7. The system according to claim 6, wherein the status diagnosis server is further configured to:
acquire a plurality of pieces of training sample data, wherein each of the plurality of pieces of training sample data comprises a set of detection data and a corresponding set of status diagnosis data, and the status diagnosis data comprises at least one of fault diagnosis data and aging diagnosis data; and
acquire the status diagnosis model by deep learning on the acquired plurality of pieces of training sample data.

8. The system according to claim 7, wherein the status diagnosis server is further configured to:
receive revised training sample data, wherein the revised training sample data comprises revised data acquired by revising the real-time status diagnosis data and the real-time detection data corresponding to the real-time status diagnosis data;
set a weight value of the revised training sample data to be greater than a predetermined weight value; and
update the status diagnosis model by deep learning on the plurality of pieces of training sample data and the revised training sample data.

9. The system according to claim 7, wherein
the radiotherapy equipment comprises various types of components;
the status diagnosis server is further configured to:
acquire training sample data of each component in at least one component by classifying the plurality of pieces of training sample data based on a type of a component corresponding to detection data in each of the plurality of pieces of training sample data; and
acquire a status diagnosis model of the each component by deep learning on the training sample data of each component; and
in response to acquiring real-time detection data of a target component, the status diagnosis server is further configured to:
generate the real-time status diagnosis data by processing the real-time detection data of the target component by using a status diagnosis model of the target component.

10. The system according to claim 7, wherein
the status diagnosis server is further configured to:
classify the plurality of pieces of training sample data based on a type of detection data in each of the plurality of pieces of training sample data;
acquire a plurality of types of status diagnosis models by deep learning on each type of training sample data;
determine a corresponding type of status diagnosis model based on a type of the acquired real-time detection data; and
generate the real-time status diagnosis data by processing the real-time detection data by using the corresponding type of status diagnosis model.

11. The system according to claim 6, wherein the radiotherapy equipment is disposed at a radiotherapy center, and the remote maintenance platform is disposed at an equipment maintenance center; and the status diagnosis server is disposed at one of the radiotherapy center, the equipment maintenance center, and a cloud computing center.

12. (canceled)

13. A device for diagnosing status of radiotherapy equipment, comprising: a processor, and a memory storing a computer program runnable by the processor, wherein the processor, when running the computer program, is caused to perform a method for diagnosing status of radiotherapy equipment, and the method comprises:
acquiring real-time detection data in an operating process of the radiotherapy equipment;
generating real-time status diagnosis data by processing the real-time detection data by using a stats diagnosis model, wherein the real-time status diagnosis data comprises at least one of real-time fault diagnosis data and real-time aging diagnosis data; and
outputting the real-time status diagnosis data.

14. A non-transitory computer-readable storage medium storing at least one instruction, wherein the non-transitory computer-readable storage medium, when running on a computer, causes the computer to perform the method for diagnosing status of radiotherapy equipment of claim 1.

15. The device according to claim 13, wherein before acquiring the real-time detection data in the operating process of the radiotherapy equipment, the method further comprises:
acquiring a plurality of pieces of training sample data, wherein each of the plurality of pieces of training sample data comprises a set of detection data and a corresponding set of status diagnosis data, and the status diagnosis data comprises at least one of fault diagnosis data and aging diagnosis data; and
acquiring the status diagnosis model by deep learning on the acquired plurality of pieces of training sample data.

16. The device according to claim 15, wherein the method further comprises:
receiving revised training sample data, wherein the revised training sample data comprises revised data acquired by revising the real-time status diagnosis data and the real-time detection data corresponding to the real-time status diagnosis data;
setting a weight value of the revised training sample data to be greater than a predetermined weight value; and
updating the status diagnosis model by deep learning on the plurality of pieces of training sample data and the revised training sample data.

17. The device according to claim 15, wherein
the radiotherapy equipment comprises various types of components;
acquiring the status diagnosis model by deep learning on the acquired plurality of pieces of training sample data comprises:
acquiring training sample data of each component in at least one component by classifying the plurality of pieces of training sample data based on a type of a component corresponding to detection data in each of the plurality of pieces of training sample data; and
acquiring a status diagnosis model of the each component by deep learning on the training sample data of each component; and
in response to acquiring real-time detection data of a target component, generating the real-time status diagnosis data by processing the real-time detection data by using the status diagnosis model comprises:
generating the real-time status diagnosis data by processing the real-time detection data of the target component by using a status diagnosis model of the target component.

18. The device according to claim 15, wherein
acquiring the status diagnosis model by deep learning on the acquired plurality of pieces of training sample data comprises:
classifying the plurality of pieces of training sample data based on a type of detection data in each of the plurality of pieces of training sample data; and
acquiring a plurality of types of status diagnosis models by deep learning on each type of training sample data; and
generating the real-time status diagnosis data by processing the real-time detection data by using the status diagnosis model comprises:
determining a corresponding type of status diagnosis model based on a type of the acquired real-time detection data; and
generating the real-time status diagnosis data by processing the real-time detection data by using the corresponding type of status diagnosis model.
